(19) United States
(12) Patent Application Publication
(10) Pub. No.: US 2021/0128781 A1
(43) Pub. Date: May 6, 2021

(54) GLASS STRUCTURE AND METHOD FOR PRODUCING THE SAME

(71) Applicant: Panasonic Intellectual Property Management Co., Ltd., Osaka (JP)

(72) Inventors: Natsuki SATO, Osaka (JP); Naoki KURIZOE, Osaka (JP); Tohru SEKINO, Osaka (JP); Sunghun CHO, Osaka (JP); Tomoyo GOTO, Osaka (JP); Yeongjun SEO, Tokyo (JP)

(21) Appl. No.: 17/086,502

(22) Filed: Nov. 2, 2020

(30) Foreign Application Priority Data
Nov. 5, 2019 (JP) 2019-200776
Nov. 5, 2019 (JP) 2019-200777

(51) Int. Cl. A61L 27/10; C03C 10/00; C03C 4/00; A61L 27/12

(52) U.S. Cl. CPC A61L 27/10; C03C 10/00; A61L 2430/02; A61L 27/12; C03C 2204/00; C03C 4/0007

(57) ABSTRACT

A glass structure includes: a plurality of glass particles, each of the glass particles including SiO₂, CaO and P₂O₅; and a bonding portion that bonds the glass particles to one another and contains hydroxyapatite, wherein at least a part of the hydroxyapatite is crystalline in the bonding portion, and wherein a porosity of the glass structure is 15% or less. A method for producing the glass structure includes: preparing a mixture by mixing a plurality of glass particles and an aqueous solution with each other, each of the glass particles including SiO₂, CaO and P₂O₅, and the aqueous solution including calcium and phosphorus and having pH of 4.0 or more; and heating and pressurizing the mixture.

### TECHNICAL FIELD

The present disclosure relates to a glass structure and a method for producing the same.

### BACKGROUND ART

A sintering method has heretofore been known as a method for producing an inorganic member made of ceramics. The sintering method is a method of obtaining a sintered body by heating an aggregate of solid powder made of an inorganic substance at a temperature lower than a melting point thereof. However, in the sintering method, it is necessary to heat the solid powder at a high temperature, so that there is a problem of a large energy consumption at the time of producing the sintered body, which results in a cost increase. Therefore, developed is a method for bonding the solid powder made of an inorganic substance at a low temperature.

Non-Patent Document 1 discloses a method for sintering bioactive glass as an inorganic substance at a low temperature. Specifically, Non-Patent Document 1 discloses that a sintered body of bioactive glass nanoparticles is obtained in such a manner that, after being added with an aqueous solution, nanoparticles of bioactive glass made of Si₂—CaO—P₂O₅ is pressurized with several hundred megapascals while being heated from room temperature to 200° C. Moreover, Non-Patent Document 1 discloses that the nanoparticles of the bioactive glass made of SiO₂—CaO—P₂O₅ can be synthesized by a sol-gel method. In such a low-temperature sintering method, since a heating temperature of the bioactive glass nanoparticles is approximately 200° C., it becomes possible to greatly reduce the energy consumption during the production.

Non-Patent Document 1: “Synthesis of Sol-Gel Derived Bioactive glass nanoparticles and Their Low-temperature Sintering”, SEKINO Toru, Jul. 22, 2018, Abstract Book of 12th International Conference on Ceramic Materials and Components for Energy and Environmental Applications (CMCEE)

### SUMMARY

However, in the sintered body of the bioactive glass nanoparticles, which is disclosed in Non-Patent Document 1, a bonding portion that couples the bioactive glass nanoparticles to one another is amorphous as a whole. Mechanical strength of the bonding portion decreases when the bonding portion is amorphous. Accordingly, there has been a problem that mechanical strength of the whole of the sintered body becomes insufficient even if the bioactive glass nanoparticles are robust.

The present disclosure has been made in consideration of such a problem as described above, which is inherent in the prior art. Then, it is an object of the present disclosure to provide a glass structure producible by the low-temperature sintering method and excellent in mechanical strength, and to provide a method for producing the glass structure.

A glass structure according to a first aspect of the present disclosure includes: a plurality of glass particles, each of the glass particles including SiO₂, CaO and P₂O₅; and a bonding portion that bonds the glass particles to one another and contains hydroxyapatite, wherein at least a part of the hydroxyapatite is crystalline in the bonding portion, and wherein a porosity of the glass structure is 15% or less.

A method for producing the glass structure according to a second aspect of the present disclosure includes: preparing a mixture by mixing a plurality of glass particles and an aqueous solution with each other, each of the glass particles including SiO₂, CaO and P₂O₅, and the aqueous solution including calcium and phosphorus and having pH of 4.0 or more; and heating and pressurizing the mixture.

### BRIEF DESCRIPTION OF THE DRAWINGS

The figures depict one or more implementations in accordance with the present teaching, by way of example only, not by way of limitations. In the figures, like reference numerals refer to the same or similar elements.

FIG. 1 is a cross-sectional view schematically illustrating an example of a glass structure according to this embodiment.

FIG. 2 is a schematic view for explaining a mechanism in which hydroxyapatite is generated on the surfaces of glass particles.

FIG. 3A is a schematic view illustrating a state in which an aqueous solution is present between adjacent glass particles in a production process of the glass structure.

FIG. 3B is a schematic view illustrating a state in which a bonding portion is formed between the adjacent glass particles.

FIG. 4 is a schematic view for explaining a mechanism in which fluorapatite and hydroxyapatite are generated on the surfaces of glass particles.

FIG. 5A is a graph illustrating X-ray diffraction patterns of test samples 1-2 to 1-6 and an X-ray diffraction pattern of hydroxyapatite registered as JCPDS 09-0432.

FIG. 5B is a graph illustrating X-ray diffraction patterns of test samples 1-1 to 1-6.

FIG. 6 is a view illustrating reflected electron images of the test samples 1-2 to 1-6 observed by a scanning electron microscope.

FIG. 7A is a graph illustrating pieces of Vickers hardness of the test samples 1-1 to 1-6.

FIG. 7B is a graph illustrating relationships between relative densities and the pieces of Vickers hardness of the test samples 1-2 to 1-6.

FIG. 8A is a graph illustrating X-ray diffraction patterns of test samples 2-1 to 2-4 and an X-ray diffraction pattern of fluorapatite registered as JCPDS 15-0876.

FIG. 8B is a graph illustrating X-ray diffraction patterns of test samples 2-1 to 2-5.

FIG. 9 is a view illustrating reflected electron images of the test samples 2-1 to 2-4 observed by a scanning electron microscope.

FIG. 10 is photographs showing results of performing a mapping analysis of fluorine for the test samples 2-1 to 2-4 by energy dispersive X-ray spectrometry (EDX).

### DETAILED DESCRIPTION

A description will be given below of a glass structure and a method for producing the glass structure according to this embodiment with reference to the drawings. Note that dimensional ratios in the drawings are exaggerated for convenience of explanation, and are sometimes different from actual ratios.

### Glass Structure of First Embodiment

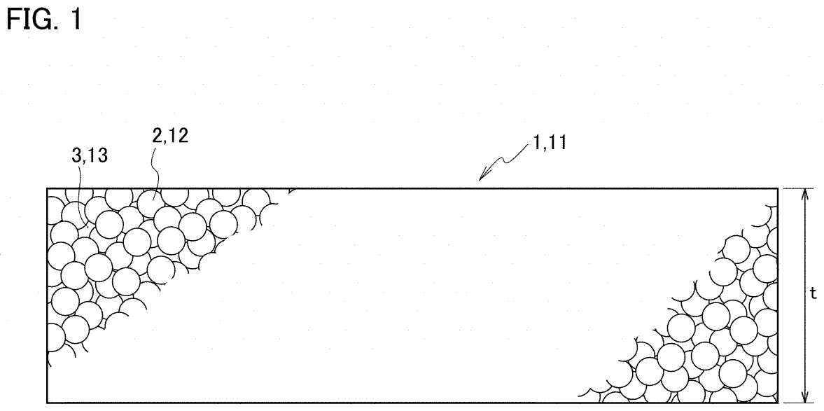

As illustrated in FIG. 1, a glass structure 1 of this embodiment includes a plurality of glass particles 2. Then, the glass particles 2 adjacent to one another are bonded to one another, whereby the glass structure 1 composed by coupling the glass particles 2 to one another is formed. Moreover, pores 3 are present between the adjacent glass particles 2.

Each of the glass particles 2 contains at least silicon dioxide (Si₂), calcium oxide (CaO) and diphosphorus pentaoxide (P₂O₅). Moreover, each of the glass particles 2 is preferably composed of bioactive glass containing at least SiO₂, CaO and P₂O₅. The bioactive glass has a property of being bonded to a bone by forming hydroxyapatite on a surface layer thereof in vivo. As such bioactive glass as described above, there can be used at least one selected from the group consisting of: SiO₂—CaO—P₂O₅ composed of SiO₂, CaO and P₂O₅; SiO₂—CaO—Na₂O—P₂O₅ composed of SiO₂, CaO, Na₂O and P₂O₅; Si₂—CaO—Na₂O—P₂O₅—K₂O—MgO composed of SiO₂, CaO, Na₂O, P₂O₅, K₂O and MgO; SiO₂—CaO—Al₂O—P₂O₅ composed of SiO₂, CaO, Al₂O and P₂O₅; and combinations thereof.

Here, a composition ratio of SiO₂ in the glass particles 2 is preferably 20% by mass or more. Moreover, a composition ratio of CaO in the glass particles 2 is preferably 20% by mass or more. Further, a composition ratio of P₂O₅ in the glass particles 2 is preferably 50% by mass or less. As will be described later, the adjacent glass particles 2 are bonded to one another via a bonding portion containing the hydroxyapatite (Ca₁₀(PO₄)₆(OH)₂). Therefore, the composition ratios of SiO₂, CaO and P₂O₅ in the glass particles 2 stay within the above-described range, whereby generation of the hydroxyapatite can be promoted by a production method to be described later.

An average particle size of the glass particles 2 which constitute the glass structure 1 is not particularly limited; however, is preferably 5 nm or more and 10 μm or less, more preferably 10 nm or more and 0.2 μm or less. The average particle size of the glass particles 2 stays within this range, whereby the glass particles 2 are strongly bonded to one another, thus making it possible to enhance strength of the glass structure 1. Moreover, the average particle size of the glass particles 2 stays within this range, whereby a ratio of the pores 3 present inside the glass structure 1 becomes 15% or less as will be described later, thus making it possible to enhance the strength of the glass structure 1. Note that, unless specifically described, as a value of the “average particle size”, a value is adopted, which is calculated as an average value of particle sizes of particles observed in several to several ten visual fields by using observation means such as a scanning electron microscope (SEM) or a transmission electron microscope (TEM).

A shape of the glass particle 2 is not particularly limited; however, can be made spherical for example. Moreover, the glass particle 2 may be particles with a polyhedral shape including a cube and a rectangular parallelepiped shape, particles with a whisker shape (needle shape), or particles with a scale leaf shape. Such polyhedral particles, whisker-shaped particles or scale leaf-shaped particles have enhanced contact property as compared to the spherical particles, and accordingly, make it possible to enhance the strength of the whole of the glass structure 1.

As mentioned above, the glass structure 1 is composed of a particle group of the glass particles 2. That is, the glass structure 1 is composed of the plurality of glass particles 2 made of the bioactive glass as a main component. The glass particles 2 are bonded to one another, whereby the glass structure 1 is formed. At this time, the glass particles 2 may be in a point contact state, or may be in a surface contact state in which particle surfaces of the glass particles 2 contact one another.

In the glass structure 1, the adjacent glass particles 2 are bonded to one another via the bonding portion containing hydroxyapatite (Ca₁₀(PO₄)₆(OH)₂). As will be described later, the glass structure 1 can be formed by heating a mixture of the glass particles 2 and an aqueous solution, which contains calcium and phosphorus and has pH of 4.0 or more, while pressurizing the mixture. Then, when the mixture is heated while being pressurized, the hydroxyapatite is generated on the surfaces of the glass particles 2, whereby the adjacent glass particles 2 can be coupled to one another by the hydroxyapatite.

Here, originally, apatite is a mineral name represented by the composition formula: M₁₀(ZO₄)₆(X)₂; however, is also used as a general term of synthetic compounds having such a composition. In the composition formula: M₁₀(ZO₄)(X)₂, at least one of alkaline earth metal and lead can be coordinated on M. Moreover, at least one selected from the group consisting of P, As, V and S can be coordinated on Z, and at least one selected from the group consisting of F, Cl, Br, OH, O and CO₃ can be coordinated on X. Therefore, in the hydroxyapatite (Ca₁₀(PO₄)(OH)₂), F⁻, Cl⁻ or CO₃²⁻ can be substituted for OH⁻ at the position of X.

As mentioned above, preferably, the bonding portion that couples the adjacent glass particles 2 to one another contains at least the hydroxyapatite, which is preferably a main component. Moreover, the bonding portion may be a region composed of the hydroxyapatite. However, as mentioned above, in the hydroxyapatite (Ca₁₀(PO₄)₆(OH)₂), F⁻, Cl⁻ or CO₃²⁻ can be substituted for OH⁻. Therefore, the bonding portion may contain apatite, for example, in which F is substituted for OH⁻ in a part of the hydroxyapatite. Moreover, besides the apatite, the bonding portion may contain a component derived from the glass particles 2, or a component derived from such an aqueous solution which contains calcium and phosphorus and has pH of 4.0 or more.

In the bonding portion of the glass structure 1, preferably, at least a part of the hydroxyapatite is crystalline. The crystalline hydroxyapatite has a property with high mechanical strength as compared to an amorphous one. Therefore, when at least a part of the hydroxyapatite contained in the bonding portion is crystalline, the strength of the bonding portion is enhanced. Therefore, the mechanical strength of the glass structure 1 can also be enhanced. The crystalline hydroxyapatite has a hexagonal crystal system or a monoclinic crystal system.

As will be described later, the mechanical strength of the glass structure 1 tends to be enhanced as a ratio of the crystalline hydroxyapatite increases in the bonding portion. Therefore, from a viewpoint of enhancing the mechanical strength, preferably, the hydroxyapatite of the bonding portion is as crystalline as possible.

A porosity on a cross section of the glass structure 1 is preferably 15% or less. That is, when the cross section of the glass structure 1 is observed, an average value of ratios of pores per unit area is preferably 15% or less. When the porosity is 15% or less, a bonding ratio of the glass particles 2 increases, so that the glass structure 1 is densified, thus making it possible to enhance the mechanical strength. Moreover, when the porosity is 15% or less, a crack is suppressed from occurring in the glass structure 1 from the pores 3 as a starting point. Accordingly, it becomes possible to enhance flexural strength of the glass structure 1. The porosity on the cross section of the glass structure 1 is more preferably 10% or less, still more preferably 5% or less. As the porosity on the cross section of the glass structure 1 is smaller, the crack that starts to occur from the pores 3 is suppressed, and accordingly, it becomes possible to enhance the strength of the glass structure 1.

In this specification, the porosity can be obtained as follows. First, the cross section of the glass structure 1 is observed, and the glass particles 2 and the pores 3 are determined. Then, the unit area and an area of the pores 3 in the unit area are determined, and the ratio of the pores 3 per unit area is obtained. Such ratios of the pores 3 per unit area are obtained at a plurality of spots, and the average value of the ratios of the pores 3 per unit area is defined as the porosity. At the time of observing the cross section of the glass structure 1, an optical microscope, a scanning electron microscope (SEM) or a transmission electron microscope (TEM) can be used. The unit area and the area of the pores 3 in the unit area may be measured by binarizing an image observed by the microscope.

A size of the pores 3 present inside the glass structure 1 is not particularly limited; however, is preferably as small as possible. The fact that the size of the pores 3 is small suppresses the crack that starts to occur from the pores 3, and accordingly, it becomes possible to enhance the mechanical strength of the glass structure 1. The size of the pores 3 of the glass structure 1 is preferably 5 μm or less, more preferably 1 μm or less, still more preferably 100 nm or less. Like the porosity mentioned above, the size of the pores 3 present inside the glass structure 1 can be obtained by observing the cross section of the glass structure 1 by a microscope.

The glass structure 1 just needs to have a structure in which the glass particles 2 are bonded to one another via the bonding portion and the porosity is 15% or less. Therefore, a shape of the glass structure 1 is not limited if the glass structure 1 has such a structure as described above. For example, the shape of the glass structure 1 can be a plate shape, a film shape, a rectangular shape, a block shape, a rod shape, and a spherical shape. When the glass structure 1 has a plate shape or a film shape, a thickness t of the glass structure 1 is not particularly limited; however, can be set to 50 μm or more for example. The glass structure 1 of this embodiment is formed by a pressurized heating method as will be described later. Therefore, the glass structure 1 with a large thickness can be obtained with ease. Note that the thickness t of the glass structure 1 can be set to 1 mm or more, and can also be set to 1 cm or more. An upper limit of the thickness t of the glass structure 1 is not particularly limited; however, can be set to 50 cm for example.

As described above, the glass structure 1 of this embodiment includes: the plurality of glass particles 2 containing SiO₂, CaO and P₂O₅; and the bonding portion 4 that bonds the glass particles 2 to one another and contains hydroxyapatite. In the bonding portion 4, at least a part of the hydroxyapatite is crystalline. Then, the porosity of the glass structure 1 is 15% or less. In the glass structure 1, the plurality of glass particles 2 are bonded to one another via the bonding portion 4 containing the hydroxyapatite, and further, at least a part of the hydroxyapatite is crystalline. Thus, the strength of the bonding portion is enhanced, so that it also becomes possible to enhance the mechanical strength of the glass structure 1. Moreover, since the porosity of the glass structure 1 is 15% or less, the glass particles 2 are arranged densely, and the mechanical strength of the glass structure 1 is enhanced. Therefore, the glass structure 1 can be provided with high machinability.

Moreover, preferably, the glass structure 1 contains a crystal phase that has diffraction peaks at diffraction angles 2θ=25.0° or more and 26.0° or less, 2θ=31.0° or more and 33.0° or less, and 2θ=39.0° or more and 40.0° or less in an X-ray diffraction pattern measured by a Cu-Kαray. When the crystal phase of the glass structure 1 has diffraction peaks within the above-described ranges as a result of X-ray diffraction measurement performed for the glass structure 1, the glass structure 1 contains the crystalline hydroxyapatite. In this case, the bonding portion contains the crystalline hydroxyapatite, thus making it possible to enhance the mechanical strength of the glass structure 1.

Preferably, the glass particles 2 have an ability to form apatite. As will be described later, the glass structure 1 can be formed by heating a mixture of the glass particles 2 and an aqueous solution, which contains calcium and phosphorus and has pH of 4.0 or more, while pressurizing the mixture. Therefore, when the glass particles 2 have a function to react with the above-described aqueous solution to form the apatite, it becomes possible to efficiently generate the bonding portion containing the hydroxyapatite, and to strongly bond the glass particles 2 to one another.

### Method for Producing Glass Structure of First Embodiment

Next, a description will be given of a method for producing the glass structure 1 according to this embodiment. The method for producing the glass structure 1 includes the steps of: preparing a mixture by mixing a plurality of glass particles 2 and an aqueous solution with each other; and heating and pressurizing the mixture. Each of the glass particles 2 contains SiO₂, CaO and P₂O₅, and the aqueous solution contains calcium and phosphorus and has pH of 4.0 or more.

The method for producing the glass structure 1 according to this embodiment is a method using a biomineral forming action in which a living body creates a mineral (inorganic compound) in a body thereof, that is, a so-called biomineralization reaction. That is, since the glass particles 2 contain CaO and P₂O₅ as components thereof as mentioned above, the glass particles 2 have a property to react with a body fluid to generate apatite on surfaces thereof. By using this mechanism, the method for producing the glass structure 1 pressurizes the above-described aqueous solution and the glass particles 2 while heating the same, and thereby reacts the aqueous solution and the glass particles 2 with each other to form the bonding portion containing the hydroxyapatite.

Specifically, first, the glass particles 2 and the aqueous solution, which contains calcium and phosphorus and has pH of 4.0 or more, are mixed with each other to prepare a mixture. A method for preparing the glass particles 2 containing SiO₂, CaO and P₂O₅ is not particularly limited; however, can be prepared by a sol-gel method, for example, by using precursors of SiO₂, CaO and P₂O₅. For example, tetraethyl orthosilicate (Si(OC₂H)₄) can be used as the precursor of SiO₂. For example, calcium nitrate tetrahydrate (Ca(NO₃)₂.4H₂O) can be used as the precursor of CaO. For example, diammonium hydrogen phosphate ((NH₄)₂HPO₄) can be used as the precursor of P₂O₅.

An average primary particle size of the glass particles 2 to be mixed with the aqueous solution is not particularly limited; however, is preferably 5 nm or more and 10 μm or less, more preferably 10 nm or more and 0.2 μm or less. The average particle size of the glass particles 2 stays within this range, whereby it becomes possible to enhance reactivity thereof with the aqueous solution to easily form the bonding portion.

For example, a simulated body fluid can be used as the aqueous solution which contains calcium and phosphorus and has pH of 4.0 or more. The simulated body fluid is an aqueous solution in which an inorganic ion concentration is set substantially equal to that of an extracellular fluid of a human body. By using this solution, an in-vivo reaction on the surface of the material can be easily predicted even on the outside of a living body. Then, the glass particles 2 containing SiO₂, CaO and P₂O₅ have a property to react with the simulated body fluid to generate the hydroxyapatite on the surfaces of the glass particles 2. Therefore, by using the simulated body fluid as such an aqueous solution, the bonding portion containing the hydroxyapatite can be formed with ease. An example of the composition of the simulated body fluid is shown in Table 1 together with the composition of human blood plasma.

TABLE 1

| | Concentration (mM) | |
|---|---|---|
| | Simulated | Blood |
| Ion | body fluid | plasma |
| Na⁺ | 142.0 | 142 |
| K⁺ | 5.0 | 5 |
| Mg²⁺ | 1.5 | 1.5 |
| Ca²⁺ | 2.5 | 7.5 |
| Cl⁻ | 148.8 | 103 |
| HCO₃⁻ | 4.2 | 27.0 |
| HPO₄²⁻ | 1.0 | 1.0 |
| SO₄²⁻ | 0.5 | 0.5 |

An addition amount of the aqueous solution to the glass particles 2 is preferably an amount by which the biomineralization reaction proceeds sufficiently. The addition amount of the aqueous solution is preferably 1 to 200% by mass, more preferably 7 to 100% by mass with respect to the glass particles 2.

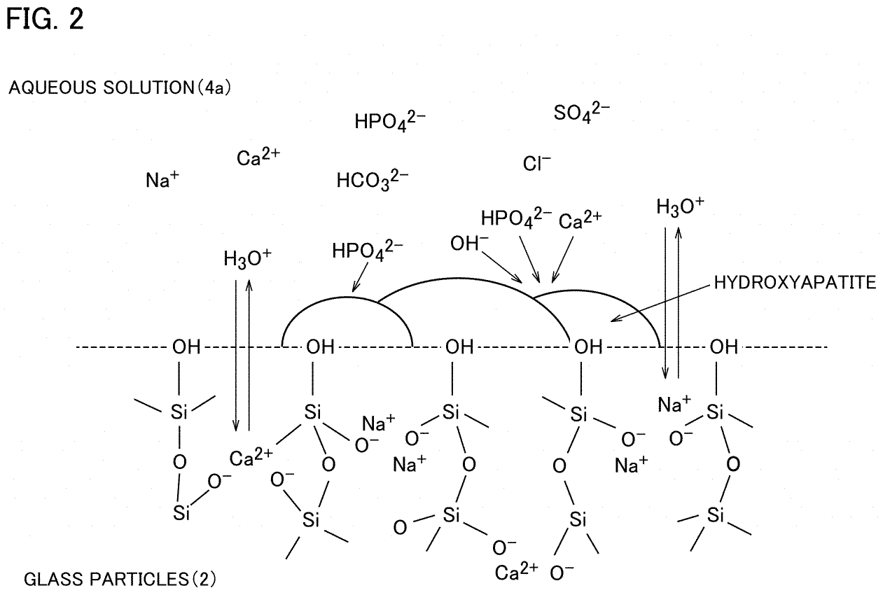

Subsequently, the inside of a metal mold is filled with the mixture composed by mixing the glass particles 2 and the aqueous solution with each other. After the metal mold is filled with the mixture, the metal mold may be heated according to needs. Then, a pressure is applied to the mixture in the inside of the metal mold, whereby a pressure in the inside of the metal mold increases. At this time, the inside of the metal mold is loaded with the glass particles 2 at a high density, and the glass particles 2 are bonded to one another, whereby a density thereof increases. That is, when the mixture composed by mixing the glass particles 2 and the aqueous solution with each other is pressurized while being heated, such a biomineralization reaction as illustrated in FIG. 2 proceeds.

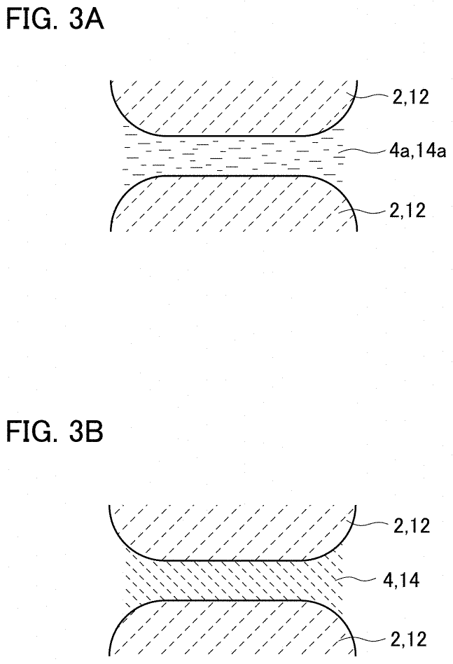

More specifically, as illustrated in FIGS. 2 and 3A, when the glass particles 2 contact the aqueous solution 4a, calcium ions (Ca²⁺) are eluted from the glass particles 2 to the aqueous solution 4a, and a large amount of silanol groups (Si—OH) are generated on the surfaces of the glass particles 2. Then, the silanol groups induce heterogeneous nucleation of the apatite, and meanwhile, the eluted Ca²⁺ raises supersaturation of the apatite in the surrounding aqueous solution 4a, and promotes nucleation of the apatite. Nuclei of the apatite, which are thus formed, take in the calcium ions and monohydrogen phosphate ions from the surrounding aqueous solution 4a, and generate apatite layers.

Thereafter, the apatite layers generated on the surfaces of the adjacent glass particles 2 are bonded to one another, whereby the bonding portion 4 containing hydroxyapatite is formed on necking portions of the adjacent glass particles 2 as illustrated in FIG. 3B. Here, a heating and pressurizing time of the mixture composed by mixing the glass particles 2 and the aqueous solution with each other is increased, whereby the crystallization of the hydroxyapatite proceeds, and the ratio of the crystalline hydroxyapatite increases. Therefore, a heating and pressurizing step of the mixture is performed for a predetermined time, whereby the bonding portion 4 containing the crystalline hydroxyapatite can be formed.

Heating and pressurizing conditions of the mixture composed by mixing the glass particles 2 and the aqueous solution with each other are not particularly limited if the reaction between the glass particles 2 and the aqueous solution proceeds under the conditions concerned. For example, preferably, the mixture composed by mixing the glass particles 2 and the aqueous solution with each other is pressurized by a pressure of 300 MPa while being heated at 40° C. or more and 300° C. or less. Moreover, preferably, a time of heating and pressurizing this mixture is 30 minutes or more. By such conditions as described above, the bonding portion 4 containing the crystalline hydroxyapatite can be formed with ease. The pressure to be applied to the mixture is preferably 1 to 1000 MPa, more preferably 10 to 500 MPa. The time of heating and pressurizing the mixture is preferably 30 minutes to 24 hours, more preferably 30 minutes to 12 hours.

Finally, a molded body is taken out of the inside of the metal mold, whereby the glass structure 1 in which the plurality of glass particles 2 are bonded to one another via the bonding portion 4 can be obtained.

As described above, the method for producing the glass structure 1 of this embodiment includes the step of preparing a mixture by mixing a plurality of glass particles 2 containing Si₂, CaO and P₂O₅ and an aqueous solution 4a, which contains calcium and phosphorus and has pH of 4.0 or more, with each other. The method for producing the glass structure 1 of this embodiment further includes the step of heating and pressurizing the mixture. A temperature of heating the mixture is preferably 40° C. or more and 300° C. or less. Moreover, a pressure to be applied to the mixture is preferably 1 MPa or more. Further, preferably, a time of heating and pressurizing the mixture is 30 minutes or more. In the production method of this embodiment, the glass structure 1 is molded under such low-temperature conditions, and accordingly, energy consumption at the time of production is reduced, thus making it possible to suppress production cost.

### Glass Structure of Second Embodiment

As illustrated in FIG. 1, a glass structure 11 of this embodiment includes a plurality of glass particles 12. Then, the glass particles 12 adjacent to one another are bonded to one another, whereby the glass structure 11 composed by coupling the glass particles 12 to one another is formed. Moreover, pores 13 are present between the adjacent glass particles 12.

For the glass particles 12, the same ones as the glass particles 2 of the first embodiment can be used. That is, each of the glass particles 12 contains at least silicon dioxide (SiO₂), calcium oxide (CaO) and diphosphorus pentaoxide (P₂O). Moreover, each of the glass particles 12 is preferably composed of bioactive glass containing at least SiO₂, CaO and P₂O₅.

Here, a composition ratio of SiO₂ in the glass particles 12 is preferably 20% by mass or more. Moreover, a composition ratio of CaO in the glass particles 12 is preferably 20% by mass or more. Further, a composition ratio of P₂O₅ in the glass particles 12 is preferably 50% by mass or less. As will be described later, the adjacent glass particles 12 are bonded to one another via a bonding portion. Further, the bonding portion contains fluorine-containing apatite derived from hydroxyapatite (Ca₁₀(PO₄)₆(OH)₂). Therefore, the composition ratios of SiO₂, CaO and P₂O₅ in the glass particles 12 stay within the above-described range, whereby generation of the hydroxyapatite and the fluorine-containing apatite derived from the same can be promoted by a production method to be described later. The glass particles 12 may contain fluorine according to needs. Thus, generation of the fluorine-containing apatite can be promoted.

An average particle size of the glass particles 12 which constitute the glass structure 11 is not particularly limited; however, like the glass particles 2 of the first embodiment, is preferably 5 nm or more and 10 μm or less, more preferably 10 nm or more and 0.2 μm or less. A shape of the glass particles 12 is not particularly limited; however, can be made similar to that of the glass particles 2 of the first embodiment.

In the glass structure 11, the adjacent glass particles 12 are bonded to one another via the bonding portion containing the fluorine-containing apatite. As will be described later, the glass structure 11 can be formed by heating a mixture of the glass particles 12 and an aqueous solution, which contains calcium, phosphorus and fluorine and has pH or 4.0 or more, while pressurizing the mixture. Then, when the mixture is heated while being pressurized, the fluorine-containing apatite is generated on the surfaces of the glass particles 12, whereby the adjacent glass particles 12 can be coupled to one another by the fluorine-containing apatite.

As mentioned above, the sintered body of Non-Patent Document 1 contains amorphous hydroxyapatite. It is known that hydroxyapatite, and particularly the amorphous hydroxyapatite is inferior in acid resistance. Therefore, if the bonding portion is composed of only the hydroxyapatite, the hydroxyapatite may sometimes be dissolved by acid, resulting in a decrease of mechanical strength of the sintered body. Hence, in the glass structure 11 of this embodiment, the bonding portion contains apatite containing fluorine. It is known that the fluorine-containing apatite is superior in acid resistance to the hydroxyapatite. Therefore, the fluorine-containing apatite is contained in the bonding portion, thus making it possible to enhance acid resistance of the bonding portion, and to also enhance acid resistance of the whole of the glass structure. As the apatite containing fluorine, mentioned can be fluorapatite (Ca₁₀(PO₄)₆(F)₂) and (Ca₁₀(PO₄)₆((OH)₁₋ₓFₓ))₂.

As mentioned above, preferably, the bonding portion that couples the adjacent glass particles 12 to one another contains at least the fluorine-containing apatite, which is preferably a main component. Moreover, the bonding portion may be a region composed of the fluorine-containing apatite. Note that, as will be described later, the fluorine-containing apatite contained in the bonding portion is generated by substituting F⁻ for OH⁻ of the hydroxyapatite (Ca₁₀(PO₄)₆(OH)₂). Therefore, the bonding portion may contain the hydroxyapatite in addition to the fluorine-containing apatite. Moreover, as mentioned above, in the hydroxyapatite (Ca₁₀(PO₄)₆(OH)₂). Cl⁻ or CO₃²⁻ can be substituted for OH⁻. Therefore, the bonding portion may contain apatite, for example, in which Cl⁻ or CO₃²⁻ is substituted for OH⁻ in a part of the hydroxyapatite. Moreover, besides the apatite, the bonding portion may contain a component derived from the glass particles 12, or a component derived from such an aqueous solution which contains calcium, phosphorus and fluorine and has pH or 4.0 or more.

In the bonding portion of the glass structure 11, the fluorine-containing apatite may be amorphous or crystalline. Regardless of a crystal structure, the fluorine-containing apatite is superior in acid resistance to the hydroxyapatite. However, in the bonding portion, preferably, at least a part of the fluorine-containing apatite is crystalline. The crystalline fluorine-containing apatite has a property with high mechanical strength as compared to an amorphous one. Therefore, when at least a part of the fluorine-containing apatite contained in the bonding portion is crystalline, the mechanical strength of the bonding portion is enhanced. Therefore, the mechanical strength of the glass structure 11 can also be enhanced.

A porosity on a cross section of the glass structure 11 is preferably 15% or less like the glass structure 1 of the first embodiment. The porosity on the cross section of the glass structure 11 is more preferably 10% or less, still more preferably 5% or less.

A size of the pores 13 present inside the glass structure 11 is not particularly limited. However, like the glass structure 1 of the first embodiment, the size of the pores 13 is preferably 5 μm or less, more preferably 1 μm or less, still more preferably 100 nm or less.

For example, like the glass structure 1 of the first embodiment, the shape of the glass structure 11 can be a plate shape, a film shape, a rectangular shape, a block shape, a rod shape, and a spherical shape. When the glass structure 11 has a plate shape or a film shape, a thickness t thereof is not particularly limited; however, can be set to 50 μm or more for example. Moreover, the thickness t of the glass structure 11 can be set to 1 mm or more, and can also be set to 1 cm or more. An upper limit of the thickness t of the glass structure 11 is not particularly limited; however, can be set to 50 cm for example.

As described above, the glass structure 11 of this embodiment includes: the plurality of glass particles 12, each of the glass particles 12 including SiO₂, CaO and P₂O₅; and the bonding portion that bonds the glass particles 12 to one another and contains apatite containing fluorine. Then, the porosity of the glass structure 11 is 15% or less. In the glass structure 11, the plurality of glass particles 12 are bonded to one another via the bonding portion 14 containing the fluorine-containing apatite. The acid resistance of the bonding portion is enhanced by the fluorine-containing apatite, so that it also becomes possible to enhance the acid resistance of the glass structure 11. Moreover, since the porosity of the glass structure 11 is 15% or less, the glass particles 12 are arranged densely, and the mechanical strength of the glass structure 11 is enhanced. Therefore, the glass structure 11 can be provided with high machinability.

Moreover, preferably, the glass structure 11 contains a crystal phase that has diffraction peaks at diffraction angles 2θ=25.0° or more and 26.0° or less, 2θ=31.00 or more and 33.0° or less, and 2θ=39.0° or more and 40.0° or less in an X-ray diffraction pattern measured by a Cu-Kα ray. When the crystal phase of the glass structure 11 has diffraction peaks within the above-described ranges as a result of X-ray diffraction measurement performed for the glass structure 11, the glass structure 11 contains the crystalline fluorine-containing apatite. In this case, the bonding portion contains the crystalline fluorine-containing apatite, thus making it possible to enhance the mechanical strength of the glass structure 11.

Preferably, the glass particles 12 have an ability to form apatite. As will be described later, the glass structure 11 can be formed by heating a mixture of the glass particles 12 and such an aqueous solution, which contains calcium, phosphorus and fluorine and has pH of 4.0 or more, while pressurizing the mixture. Therefore, when the glass particles 12 have a function to react with the above-described aqueous solution to form the apatite, it becomes possible to efficiently generate the bonding portion containing the fluorine-containing apatite, and to strongly bond the glass particles 12 to one another.

### Method for Producing Glass Structure of Second Embodiment

Next, a description will be given of a method for producing the glass structure 11 according to this embodiment. The method for producing the glass structure 11 includes the steps of: preparing a mixture by mixing a plurality of glass particles 12 and an aqueous solution with each other; and heating and pressurizing the mixture. Each of the glass particles 2 contains SiO₂, CaO and P₂O₅, and the aqueous solution contains calcium, phosphorus and fluorine and has pH of 4.0 or more.

The method for producing the glass structure 11 according to this embodiment is a method using a biomineral forming action in which a living body creates a mineral (inorganic compound) in a body thereof, that is, a so-called biomineralization reaction. That is, since the glass particles 12 contain CaO and P₂O₅ as components thereof as mentioned above, the glass particles 12 have a property to react with a body fluid to generate apatite on surfaces thereof. By using this mechanism, the method for producing the glass structure 11 pressurizes the above-described aqueous solution and the glass particles 12 while heating the same, and thereby reacts the aqueous solution and the glass particles 12 with each other to form the bonding portion containing the fluorine-containing apatite derived from the hydroxyapatite.

Specifically, first, the glass particles 12 and the aqueous solution, which contains calcium, phosphorus and fluorine and has pH of 4.0 or more, are mixed with each other to prepare a mixture. A method for preparing the glass particles 12 containing SiO₂, CaO and P₂O₅ is not particularly limited; however, can be prepared by a sol-gel method, for example, by using precursors of SiO₂, CaO and P₂O₅.

An average primary particle size of the glass particles 12 to be mixed with the aqueous solution is not particularly limited; however, is preferably 5 nm or more and 10 μm or less, more preferably 10 nm or more and 0.2 μm or less. The average particle size of the glass particles 12 stays within this range, whereby it becomes possible to enhance reactivity thereof with the aqueous solution to easily form the bonding portion.

For example, a simulated body fluid containing fluorine can be used as the aqueous solution, which contains calcium, phosphorus and fluorine and has pH of 4.0 or more. The glass particles 12 containing SiO₂, CaO and P₂O₅ have a property to react with the simulated body fluid to generate the hydroxyapatite on the surfaces of the glass particles 12 concerned. Therefore, the hydroxyapatite can be generated with ease by using the simulated body fluid as such an aqueous solution. An example of the composition of the simulated body fluid is shown in Table 2 together with the composition of human blood plasma.

Here, the simulated body fluid does not contain fluorine. Therefore, in the production method of this embodiment, as the above-described aqueous solution, preferably used is such a simulated body fluid caused to contain fluorine therein in such a manner that fluorine ions (F) are substituted for a part of chloride ions (Cl⁻) in the above-described simulated body fluid. An example of a composition of the simulated body fluid containing fluorine is shown in Table 2 together with those of the above-described simulated body fluid and the blood plasma.

TABLE 2

| | Concentration (mM) | | |
|---|---|---|---|
| | Simulated | Fluorine-containing | Blood |
| Ion | body fluid | simulated body fluid | plasma |
| Na⁺ | 142.0 | 142.0 | 142 |
| K⁺ | 5.0 | 5.0 | 5 |
| Mg²⁺ | 1.5 | 1.5 | 1.5 |
| Ca²⁺ | 2.5 | 2.5 | 2.5 |
| Cl⁻ | 148.8 | 147.7 | 103 |
| F⁻ | — | 1.1 | — |
| HCO₃⁻ | 4.2 | 4.2 | 27.0 |
| HPO₄²⁻ | 1.0 | 1.0 | 1.0 |
| SO₄²⁻ | 0.5 | 0.5 | 0.5 |

An addition amount of the aqueous solution to the glass particles 12 is preferably an amount by which the biomineralization reaction proceeds sufficiently. The addition amount of the aqueous solution is preferably 1 to 200% by mass, more preferably 7 to 100% by mass with respect to the glass particles 12.

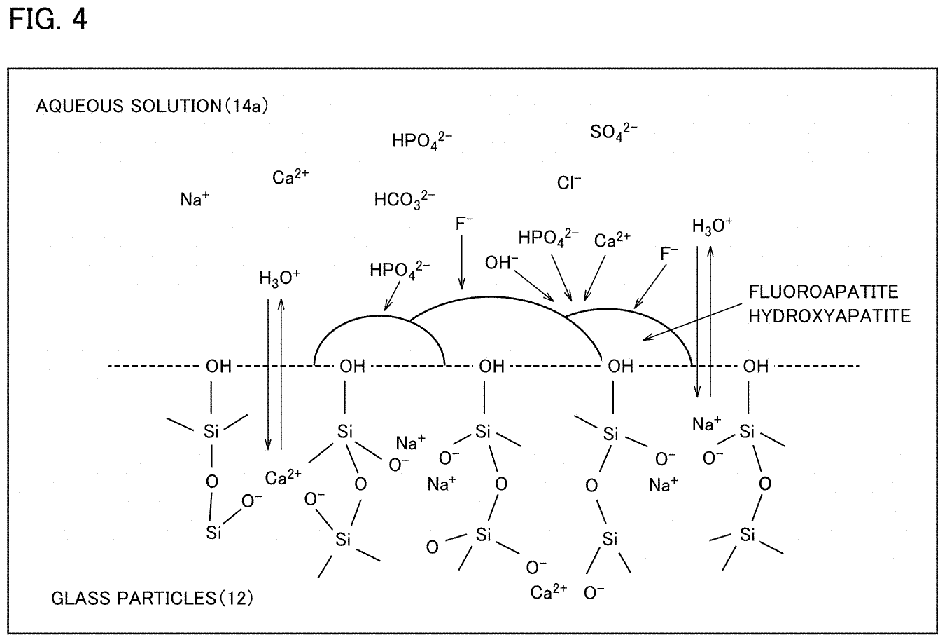

Subsequently, the inside of a metal mold is filled with the mixture composed by mixing the glass particles 12 and the aqueous solution with each other. After the metal mold is filled with the mixture, the metal mold may be heated according to needs. Then, a pressure is applied to the mixture in the inside of the metal mold, whereby a pressure in the inside of the metal mold increases. At this time, the inside of the metal mold is loaded with the glass particles 12 at a high density, and the glass particles 12 are bonded to one another, whereby a density thereof increases. That is, when the mixture composed by mixing the glass particles 12 and the aqueous solution with each other is pressurized while being heated, such a biomineralization reaction as illustrated in FIG. 4 proceeds.

More specifically, as illustrated in FIGS. 3A and 4, when the glass particles 12 contact the aqueous solution 14a, calcium ions (Ca²⁺) are eluted from the glass particles 12 to the aqueous solution 14a, and a large amount of silanol groups (Si—OH) are generated on the surfaces of the glass particles 12. Then, the silanol groups induce heterogeneous nucleation of the apatite, and meanwhile, the eluted Ca²⁺ raises supersaturation of the apatite in the surrounding aqueous solution 14a, and promotes nucleation of the apatite. Nuclei of the apatite, which are thus formed, take in the calcium ions and monohydrogen phosphate ions from the surrounding aqueous solution 14a, and generate apatite layers.

Here, F contained in the simulated body fluid is easily substituted for OH⁻ of the hydroxyapatite (Ca₁₀(PO₄)₆(OH)₂) contained in the apatite layers. Therefore, the hydroxyapatite in the apatite layers turns to the fluorine-containing apatite, that is, fluorapatite (Ca₁₀(PO₄)(F)₂).

Thereafter, the apatite layers generated on the surfaces of the adjacent glass particles 12 are bonded to one another, whereby the bonding portion 14 containing the fluorine-containing apatite is formed on necking portions of the adjacent glass particles 12 as illustrated in FIG. 3B. Here, a heating and pressurizing time of the mixture composed by mixing the glass particles 12 and the aqueous solution with each other is increased, whereby the crystallization of the fluorine-containing apatite proceeds, and the ratio of the crystalline fluorine-containing apatite increases. Therefore, a heating and pressurizing step of the mixture is performed for a predetermined time, whereby the bonding portion 14 containing the crystalline fluorine-containing apatite can be formed.

Heating and pressurizing conditions of the mixture composed by mixing the glass particles 12 and the aqueous solution with each other are not particularly limited if the reaction between the glass particles 2 and the aqueous solution proceeds under the conditions concerned. For example, preferably, the mixture composed by mixing the glass particles 12 and the aqueous solution with each other is pressurized by a pressure of 1 MPa while being heated at 40° C. or more and 300° C. or less. Moreover, preferably, a time of heating and pressurizing this mixture is 10 minutes or more. By such conditions as described above, the bonding portion 14 containing the fluorine-containing apatite can be formed with ease. The pressure to be applied to the mixture is preferably 1 to 1000 MPa, more preferably 10 to 500 MPa. The time of heating and pressurizing the mixture is preferably 1 minute to 24 hours, more preferably 30 minutes to 12 hours.

Finally, a molded body is taken out of the inside of the metal mold, whereby the glass structure 11 in which the plurality of glass particles 12 are bonded to one another via the bonding portion 14 can be obtained.

As described above, the method for producing the glass structure 11 of this embodiment includes the step of preparing a mixture by mixing a plurality of glass particles 12 containing SiO₂, CaO and P₂O₅ and an aqueous solution, which contains calcium, phosphorus and fluorine and has pH of 4.0 or more, with each other. The method for producing the glass structure 11 of this embodiment further includes the step of heating and pressurizing the mixture. A temperature of heating the mixture is preferably 40° C. or more and 300° C. or less. Moreover, a pressure to be applied to the mixture is preferably 1 MPa or more. Further, preferably, a time of heating and pressurizing the mixture is 10 minutes or more. In the production method of this embodiment, the glass structure 11 is molded under such low-temperature conditions, and accordingly, energy consumption at the time of production is reduced, thus making it possible to suppress production cost.

[Member Provided with Glass Structure]Next, a description will be given of a member including each of the glass structures 1 and 11. The glass structure 1 of the first embodiment and the glass structure 11 of the second embodiment can be formed into a plate shape with a large thickness as mentioned above. Moreover, the glass structure 1 and 11 have high mechanical strength, and can be cut in the same way as a general ceramic member, and in addition, can also be subjected to surface treatment. Therefore, each of the glass structures 1 and 11 can be suitably used as a building member. As the building member, for example, an outer wall material (siding), a roof material and the like can be mentioned though the building member is not particularly limited. Moreover, a road material and a ditch material can also be mentioned as the building member.

Generally, the hydroxyapatite has translucency. Therefore, the glass structures 1 and 11 have translucency in the bonding portions 4 and 14 in some cases. When the bonding portions 4 and 14 have translucency, it also becomes possible to enhance designability of the whole of the glass structure.

Hereinafter, the glass structure of the first embodiment will be described more in detail by Example 1, and the glass structure of the second embodiment will be described more in detail by Example 2; however, the present disclosure is not limited to these.

### Example 1

(Fabrication of Test Sample)

First, nanoparticles of three-component system bioactive glass composed of SiO₂—CaO—P₂O₅ were prepared. Specifically, tetraethyl orthosilicate (Si(OC₂Hs)₄), calcium nitrate tetrahydrate (Ca(NO₃)₂.4H₂O) and diammonium hydrogen phosphate ((NH₄)₂HPO₄) were prepared as precursors. Next, 9.167 g of tetraethyl orthosilicate and 7.639 g of calcium nitrate tetrahydrate were added to a mixed solution of ultrapure water and ethanol. A ratio of the ultrapure water and ethanol in the mixed solution was set to 2 mol:1 mol. Then, this mixed solution was poured into ultrapure water containing 1.087 g of diammonium hydrogen phosphate, and a mixed solution thus obtained was stirred for 48 hours while pH thereof was being adjusted to 11 by ammonia water. Thereafter, the mixed solution was aged for 24 hours, and was subjected to centrifugal separation, whereby a white gel was obtained. Next, the obtained gel was mixed with 6000 g/mol of a polyethylene glycol aqueous solution (1% (w/v)), and a resultant was freeze-dried. Finally, gel powder obtained after such a freeze-dry process was calcined at 700° C., whereby bioactive glass nanoparticles were obtained. An average primary particle size of the bioactive glass nanoparticles synthesized by this method was approximately 28 nm as a result of an analysis by a transmission electron microscope.

Next, 0.25 g of the bioactive glass nanoparticles and a simulated body fluid were mixed with each other to obtain a mixture. The simulated body fluid contains components shown in Table 1, and was mixed with the bioactive glass nanoparticles so that a mass percent of the simulated body fluid became 43% by mass with respect to the bioactive glass nanoparticles.

Subsequently, the obtained mixture was poured into a cylindrical metal mold having a space therein. Then, the mixture was heated and pressurized under conditions of 120° C. and 300 MPa. A heating and pressurizing time in this case was set to 10 minutes, 0.5 hours, 1.0 hour, 2.0 hours, 6.0 hours and 12.0 hours. Here, a sample heated and pressurized for 10 minutes was defined as a test sample 1-1, a sample heated and pressurized for 0.5 hours was defined as a test sample 1-2, and a sample heated and pressurized for 1.0 hour was defined as a test sample 1-3. Moreover, a sample heated and pressurized for 2.0 hours was defined as a test sample 1-4, a sample heated and pressurized for 6.0 hours was defined as a test sample 1-5, and a sample heated and pressurized for 12.0 hours was defined as a test sample 1-6. In this way, the test samples 1-1 to 1-6 different in heating and pressurizing time from one another were obtained.

(Evaluation)

<X-Ray Diffraction Measurement>

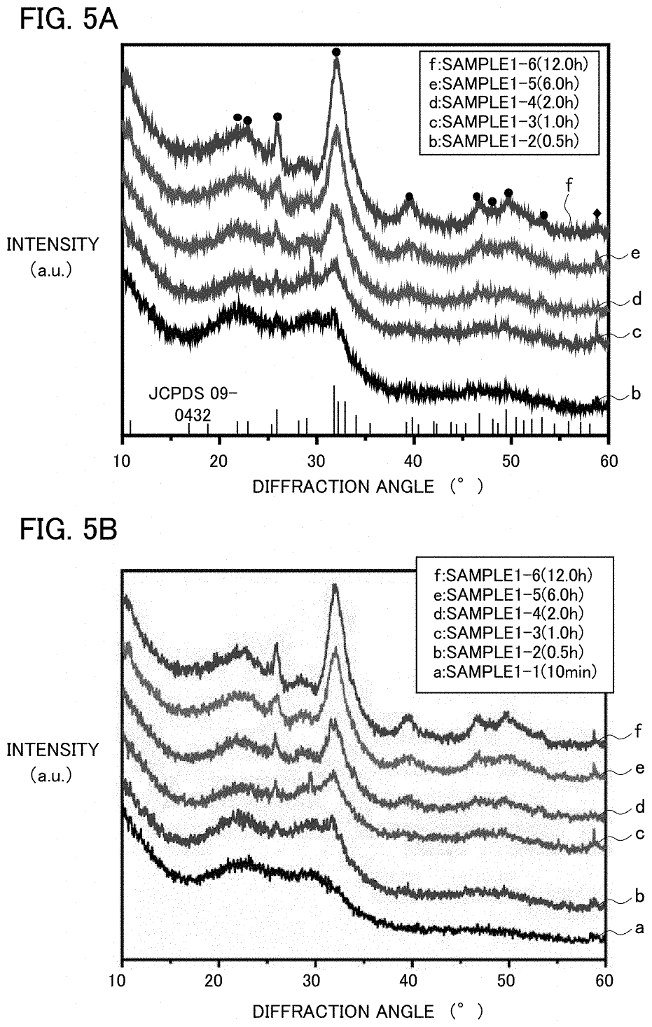

For the test samples 1-1 to 1-6 obtained as mentioned above, X-ray diffraction patterns were measured by using an X-ray diffractometer. D8 ADVANCE, an X-ray diffractometer made by BrukerAXS, was used as the X-ray diffractometer. The X-ray diffraction patterns were measured under conditions where a tube voltage was 40 kV, a tube current was 40 mA, a diffraction angle 2θ was 10° to 60°, and a step size was 0.02°. FIG. 5A illustrates the X-ray diffraction patterns of the test samples 1-2 to 1-6 and an X-ray diffraction pattern of hydroxyapatite registered as JCPDS 09-0432. FIG. 5B illustrates the X-ray diffraction patterns of the test samples 1-1 to 1-6.

As illustrated in FIG. 5A, in the test samples 1-2 to 1-6, diffraction peaks derived from the hydroxyapatite were observed. Specifically, in the test samples 1-2 to 1-6, the diffraction peaks were observed at diffraction angles 2θ=25.0° or more and 26.0° or less, 2θ=31.0° or more and 33.0° or less, and 2θ=39.0° or more and 40.0° or less in X-ray diffraction patterns measured by a Cu-Kα ray. Therefore, it can be seen that the test samples 1-2 to 1-6 contain the crystalline hydroxyapatite.

Note that, from FIG. 5B, in the test sample 1-1 heated and pressurized for 10 minutes, the diffraction peak derived from the hydroxyapatite was not able to be observed. Therefore, it can be seen that the test sample 1-1 does not contain the crystalline hydroxyapatite.

<Relative Density Measurement>

Relative densities were measured for the test samples 1-2 to 1-6. The relative densities were defined as values obtained by dividing density values of the test samples, which are measured by the Archimedes method, by a density of the bioactive glass. As a result, the relative density of the test sample 1-2 was 85%, the relative density of the test sample 1-3 was 87%, the relative density of the test sample 1-4 was 89%, the relative density of the test sample 1-5 was 92%, and the relative density of the test sample 1-6 was 93%. That is, it can be seen that, as the heating and pressurizing time of the test samples becomes longer, the relative densities of the test samples are increased, resulting in denser structures.

<Observation by Scanning Electron Microscope>

First, by using an osmium plasma coater, amorphous osmium-metal coating films were formed on the cross sections of the test samples 1-2 to 1-6. The osmium plasma coater OPC-60A made by The SPI Supplies Division of Structure Probe, Inc. was used as the osmium plasma coater. Next, by using a scanning electron microscope (SEM), reflected electron images of the cross sections of the test samples 1-2 to 1-6, on which the osmium-metal coating films were formed, were observed. Note that, as the scanning electron microscope, used was the ultra-high resolution field-emission scanning electron microscope SU9000 made by Hitachi High-Tech Corporation, in which an accelerating voltage was set to 30 keV.

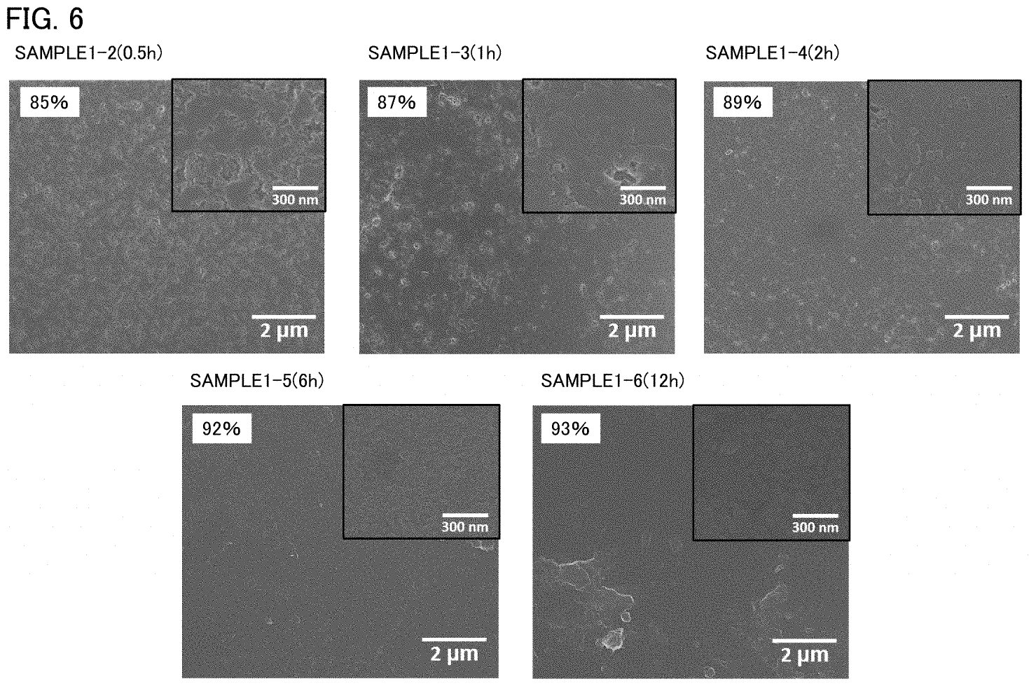

FIG. 6 shows the reflected electron images of the test samples 1-2 to 1-6, in which enlarged photographs are shown on upper right corners. Moreover, the relative densities of the respective test samples are shown on upper left corners of the respective images. From FIG. 6, it can be seen that, as the heating and pressurizing time of each of the test samples becomes longer, the number of pores present on the cross section of the test sample is reduced, resulting in a denser structure.

Moreover, area ratios of pores were calculated from the reflected electron images of the test samples 1-2 to 1-6 in FIG. 6, and the porosities thereof were obtained. As a result, all of the porosities of the test samples 1-2 to 1-6 were 15% or less.

<Vickers Hardness Measurement>

Vickers hardness was measured for each of the test samples 1-1 to 1-6. Specifically, for each test sample, a test was performed six times under conditions where a load (test force) was 19.8 N and a holding time was 15 seconds, and an average value of test results was defined as the Vickers hardness of each test sample. The Vickers hardness was measured by using the Vickers hardness meter FV-310e made by Future-Tech Corporation.

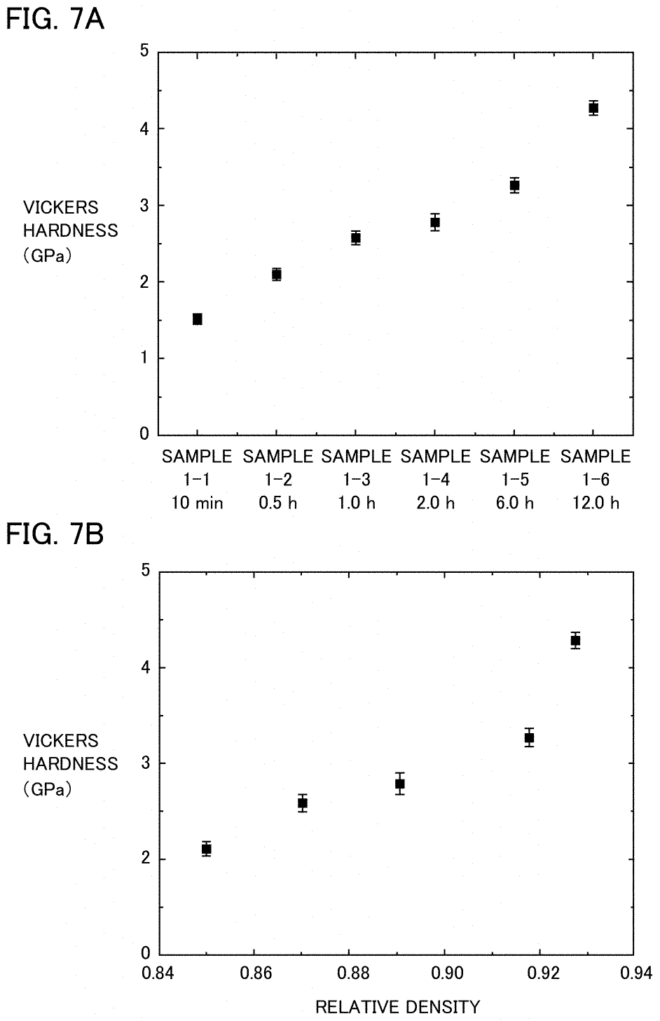

Pieces of the Vickers hardness in the respective test samples are collectively shown in Table 3 and FIG. 7A. The heating and pressurizing time of each test sample is also shown in Table 3 and FIG. 7A. It can be seen that, as shown in Table 3 and FIG. 7A, each of the test samples 1-2 to 1-6 containing the crystalline hydroxyapatite has large Vickers hardness and high mechanical strength as compared to the test sample 1-1 containing the amorphous hydroxyapatite. Moreover, it can be seen that, as a heating and pressurizing time of the mixture becomes longer, the Vickers hardness of the test sample is also increased. Further, it can be seen that, as shown in FIG. 7B, as the relative density of the test sample is increased, the Vickers hardness thereof is also increased.

TABLE 3

| | Heating and | Vickers |
|---|---|---|
| | pressurizing | hardness |
| | time | (GPa) |
| Test sample 1-1 | 10 min. | 1.521 ± 0.065 |
| Test sample 1-2 | 0.5 hrs. | 2.098 ± 0.071 |
| Test sample 1-3 | 1.0 hr. | 2.581 ± 0.089 |
| Test sample 1-4 | 2.0 hrs. | 2.785 ± 0.113 |
| Test sample 1-5 | 6.0 hrs. | 3.271 ± 0.105 |
| Test sample 1-6 | 12.0 hrs. | 4.283 ± 0.095 |

It can be seen that, as described above, the mechanical strength of the glass structure is enhanced in such a manner that the glass structure contains the crystalline hydroxyapatite. Moreover, it can be seen that, as a heating and pressurizing time of the mixture of the bioactive glass nanoparticles and the simulated body fluid becomes longer, the relative density of the glass structure is enhanced, and the crystallization of the hydroxyapatite proceeds, so that the mechanical strength of the glass structure is also enhanced.

### Example 2

<Fabrication of Test Samples 2-1 to 2-4>

First, nanoparticles of three-component system bioactive glass composed of SiO₂—CaO—P₂O₅ were prepared. Specifically, tetraethyl orthosilicate (Si(OC₂Hs)₄), calcium nitrate tetrahydrate (Ca(NO₃)₂.4H₂O) and diammonium hydrogen phosphate ((NH₄)₂HPO₄) were prepared as precursors. Next, 9.167 g of tetraethyl orthosilicate and 7.639 g of calcium nitrate tetrahydrate were added to a mixed solution of ultrapure water and ethanol. A ratio of the ultrapure water and ethanol in the mixed solution was set to 2 mol:1 mol. Then, this mixed solution was poured into ultrapure water containing 1.087 g of diammonium hydrogen phosphate, and a mixed solution thus obtained was stirred for 48 hours while pH thereof was being adjusted to 11 by ammonia water. Thereafter, the mixed solution was aged for 24 hours, and was subjected to centrifugal separation, whereby a white gel was obtained. Next, the obtained gel was mixed with 6000 g/mol of a polyethylene glycol aqueous solution (1% (w/v)), and a resultant was freeze-dried. Finally, gel powder obtained after such a freeze-dry process was calcined at 700° C., whereby bioactive glass nanoparticles were obtained. An average primary particle size of the bioactive glass nanoparticles synthesized by this method was approximately 28 nm as a result of an analysis by a transmission electron microscope.

Next, 0.3 g of the bioactive glass nanoparticles and a fluorine-containing simulated body fluid were mixed with each other to obtain a mixture. The fluorine-containing simulated body fluid was prepared by mixing distilled water, respective reagents of NaCl, NaHCO₃, KCl, K₂HPO₄.3H₂O, MgCl₂.6H₂O, CaCl₂), Na₂SO₄ and KF, a pH-adjusting tris(hydroxymethyl)aminomethane ((CH₂OH)₃CNH₂) buffer agent, and 1 M of hydrochloric acid (HCl) with one another so that these make a composition shown in Table 2. Moreover, the fluorine-containing simulated body fluid was mixed with the bioactive glass nanoparticles so that a mass percent of the fluorine-containing simulated body fluid became 43% by mass with respect to the bioactive glass nanoparticles.

Subsequently, the obtained mixture was poured into a cylindrical metal mold having a space therein. Then, the mixture was heated and pressurized under conditions of 120° C. and 300 MPa. A heating and pressurizing time in this case was set to 0.5 hours, 1.0 hour, 2.0 hours and 6.0 hours. Here, a sample heated and pressurized for 0.5 hours was defined as a test sample 2-1, and a sample heated and pressurized for 1.0 hour was defined as a test sample 2-2. Moreover, a sample heated and pressurized for 2.0 hours was defined as a test sample 2-3, and a sample heated and pressurized for 6.0 hours was defined as a test sample 2-4. In this way, the test samples 2-1 to 2-4 different in heating and pressurizing time from one another were obtained.

<Fabrication of Test Sample 2-5>

First, in a similar way to the above, bioactive glass nanoparticles with an average primary particle size of approximately 28 nm were prepared.

Next, 0.3 g of the bioactive glass nanoparticles and a simulated body fluid that does not contain fluorine were mixed with each other to obtain a mixture. The simulated body fluid that does not contain fluorine contains components shown in Table 2, and was mixed with the bioactive glass nanoparticles so that a mass percent of the simulated body fluid became 40% by mass with respect to the bioactive glass nanoparticles.

Subsequently, the obtained mixture was poured into a cylindrical metal mold having a space therein. Then, the mixture was heated and pressurized under conditions of 120° C., 300 MPa and 0.5 hours. In this way, a test sample 2-5 that did not contain fluorine was obtained.

(Evaluation)

<X-Ray Diffraction Measurement>

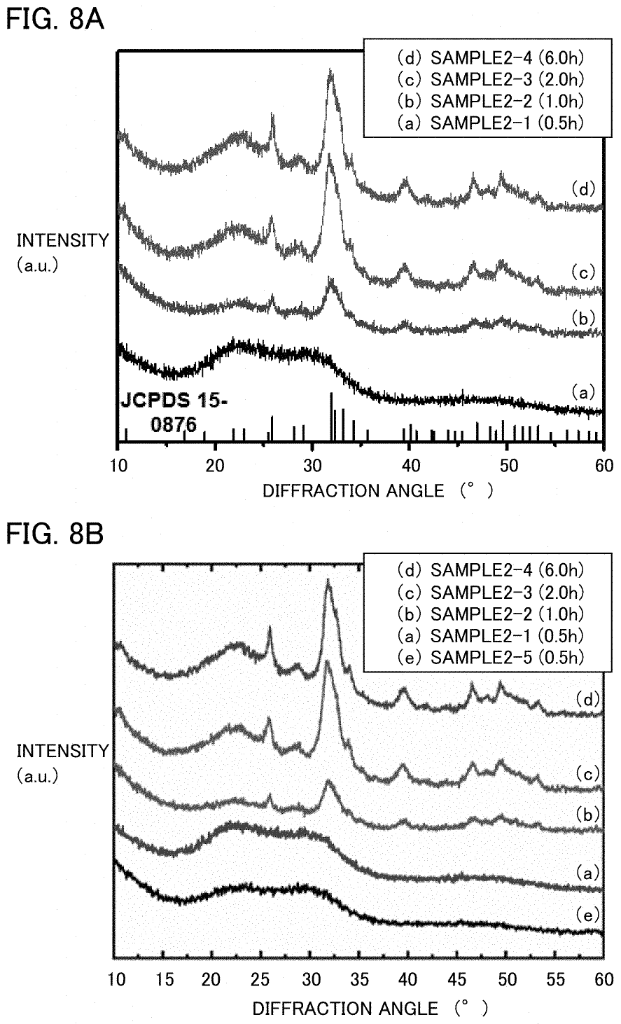

For the test samples 2-1 to 2-5 obtained as mentioned above, X-ray diffraction patterns were measured by using an X-ray diffractometer. D8 ADVANCE, an X-ray diffractometer made by BrukerAXS, was used as the X-ray diffractometer. The X-ray diffraction patterns were measured under conditions where a tube voltage was 40 kV, a tube current was 40 mA, a diffraction angle 2θ was 10° to 60°, and a step size was 0.02°. FIG. 8A illustrates the X-ray diffraction patterns of the test samples 2-1 to 2-4 and an X-ray diffraction pattern of fluorapatite registered as JCPDS 15-0876. FIG. 8B illustrates the X-ray diffraction patterns of the test samples 2-1 to 2-5.

As illustrated in FIG. 8A, in the test samples 2-2 to 2-4, diffraction peaks derived from the fluorapatite were observed. Specifically, in the test samples 2-2 to 2-4, the diffraction peaks were observed at diffraction angles 2θ=25.0° or more and 26.0° or less, 2θ=31.0° or more and 33.0° or less, and 2θ=39.0° or more and 40.0° or less in X-ray diffraction patterns measured by a Cu-Kα ray. Therefore, it can be seen that the test samples 2-2 to 2-4 contain the crystalline fluorapatite.

Note that, in the test sample 2-1 heated and pressurized for 0.5 hours, the diffraction peak derived from the fluorapatite was not able to be observed. Therefore, it can be seen that the test sample 2-1 does not contain the crystalline fluorapatite.

Note that, from FIG. 8B, in the test sample 2-5 heated and pressurized for 0.5 hours, the diffraction peak derived from the fluorapatite was not able to be observed, and further, diffraction peaks of other components were not able to be observed, either. Therefore, it can be seen that the test sample 2-5 does not contain at least crystalline apatite.

<Relative Density Measurement>

Relative densities were measured for the test samples 2-1 to 2-4. The relative densities were defined as values obtained by dividing density values of the test samples, which are measured by the Archimedes method, by a density of the bioactive glass. As a result, the relative density of the test sample 2-1 was 82%, the relative density of the test sample 2-2 was 85%, the relative density of the test sample 2-3 was 88%, and the relative density of the test sample 2-4 was 89%. That is, it can be seen that, as the heating and pressurizing time of the test samples becomes longer, the relative densities of the test samples are increased, resulting in denser structures.

<Observation by Scanning Electron Microscope>

First, by using an osmium plasma coater, amorphous osmium-metal coating films were formed on cross sections of the test samples 2-1 to 2-4. The osmium plasma coater OPC-60A made by The SPI Supplies Division of Structure Probe, Inc. was used as the osmium plasma coater. Next, by using a scanning electron microscope (SEM), reflected electron images of the cross sections of the test samples 2-1 to 2-4, on which the osmium-metal coating films were formed, were observed. Note that, as the scanning electron microscope, used was the ultra-high resolution field-emission scanning electron microscope SU9000 made by Hitachi High-Tech Corporation, in which an accelerating voltage was set to 30 keV.

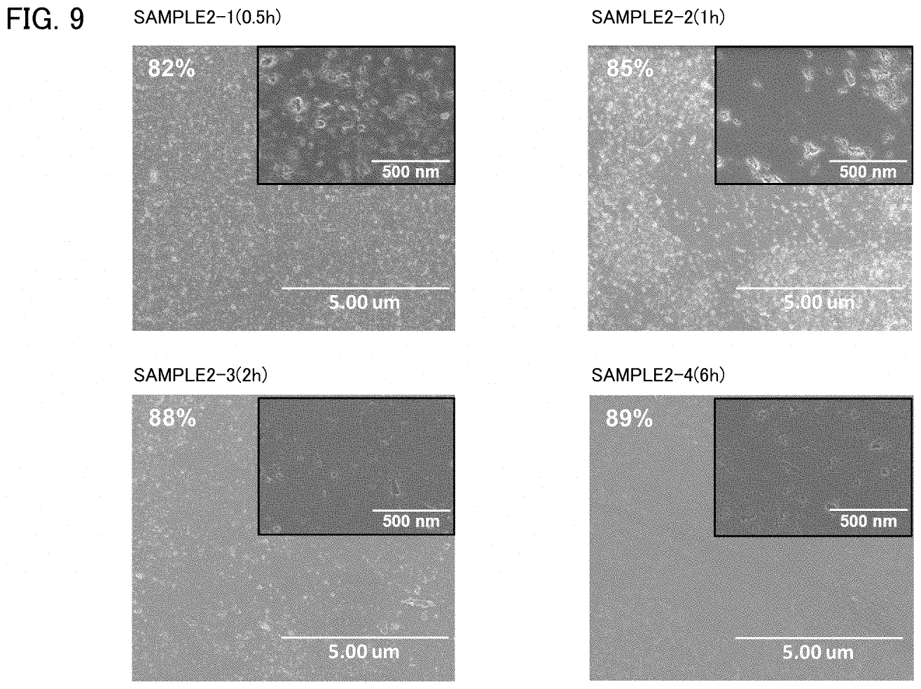

FIG. 9 shows the reflected electron images of the test samples 2-1 to 2-4, in which enlarged photographs are shown on upper right corners. Moreover, the relative densities of the respective test samples are shown on upper left corners of the respective images. From FIG. 9, it can be seen that, as the heating and pressurizing time of each of the test samples becomes longer, the number of pores present on the cross section of the test sample is reduced, resulting in a denser structure.

Moreover, area ratios of pores were calculated from the reflected electron images of the test samples 2-1 to 2-4 in FIG. 9, and the porosities thereof were obtained. As a result, all of the porosities of the test samples 2-1 to 2-4 were 15% or less.

<Component Analysis by Energy Dispersive X-Ray Spectrometry (EDX)>

For the test samples 2-1 to 2-5, elementary analysis was performed by the energy dispersive X-ray spectrometry. One made by Horiba, Ltd. was used as an energy dispersive X-ray spectrometer. Results of the elementary analysis for the test samples 2-1 to 2-5 are shown in Table 4.

TABLE 4

| | Test | Test | Test | Test | Test |
|---|---|---|---|---|---|
| | sample | sample | sample | sample | sample |
| Com- | 2-1 (0.5 h) | 2-2 (1.0 h) | 2-3 (2.0 h) | 2-4 (6.0 h) | 2-5 (0.5 h) |
| ponent | (mass %) | (mass %) | (mass %) | (mass %) | (mass %) |
| O | 53.5 | 56.4 | 54.2 | 55.1 | 55 |
| Si | 24.4 | 24.2 | 24.2 | 23.4 | 22.1 |
| Ca | 16 | 12.7 | 15.4 | 15.8 | 16.8 |
| P | 5.6 | 6.1 | 5.8 | 5.4 | 6 |
| F | 0.4 | 0.5 | 0.3 | 0.2 | 0 |

As shown in Table 4, the test samples 2-2 to 2-4 contain fluorine, and further, diffraction peaks of fluorapatite are observed therein as mentioned above. Accordingly, it can be seen that bonding portions of these test samples contain crystalline fluorapatite. Further, the test sample 2-1 also contains fluorine, and therefore, it is surmised that a bonding portion of the test sample 2-1 contains amorphous fluorapatite by the mechanism mentioned above.

Note that, from the fact that the test sample 2-5 does not contain fluorine as shown in Table 4, it can be seen that the bonding portion thereof does not contain fluorapatite.

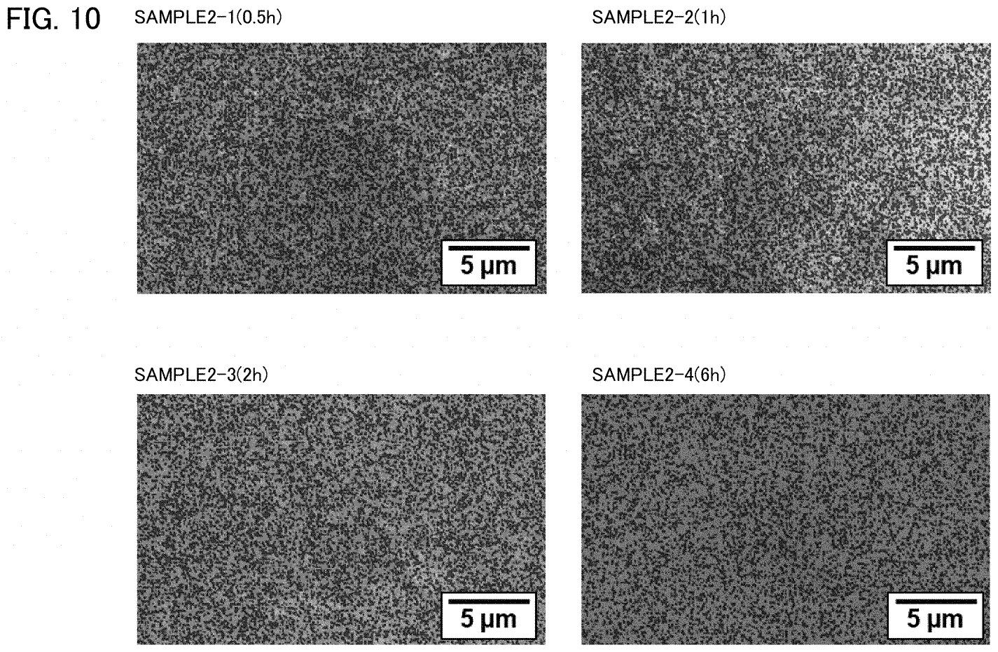

FIG. 10 shows results of performing a mapping analysis of fluorine for the test samples 2-1 to 2-4 by energy dispersive X-ray spectrometry (EDX). From FIG. 10, it can be seen that fluorine atoms are highly dispersed on the surfaces of all of the test samples 2-1 to 2-4.

As described above, since the test samples 2-1 to 2-4 according to this embodiment contain fluorapatite excellent in acid resistance, it can be seen that glass structures according to the test samples 2-1 to 2-4 are excellent in acid resistance. Moreover, since the test samples 2-2 to 2-4 contain the crystalline fluorapatite, it can be seen that the mechanical strength of each of the glass structures according thereto is also enhanced. Moreover, it can be seen that, as the heating and pressurizing time of the mixture of the bioactive glass nanoparticles and the fluorine-containing simulated body fluid becomes longer, the relative density of the glass structure is enhanced, and the crystallization of the fluorapatite proceeds, so that the mechanical strength of the glass structure is also enhanced.

<Acid Resistance Test>

An acid resistance test was performed for the test samples 2-4 and 2-5. Specifically, by the following method, elution amounts of the components with respect to acid were compared between the test sample 2-4 and the test sample 2-5.

First, 6 mL of a hydrochloric acid aqueous solution with a concentration of 3% was put into a plastic container with a capacity of 30 mL, and thereafter, a piece of each test sample was immersed into the hydrochloric acid aqueous solution. Then, after the piece of the test sample was immersed for 1 hour at room temperature, the piece of the test sample was taken out from the hydrochloric acid aqueous solution.

Next, such a solution from which the piece of the test sample was taken out was diluted with 18 mL of ion exchange water, and thereafter, ion concentrations of calcium (Ca) and phosphorus (P) in the solution were measured by the inductive coupling plasma emission analysis method (ICP-AES). Then, elution amounts of calcium (Ca) and phosphorus (P) with respect to the hydrochloric acid aqueous solution were obtained from the test samples. For the measurement by the ICP-AES, the inductively coupled plasma atomic emission spectrometer iCAP7400 Duo made by Thermo Fisher Scientific was used. Table 5 shows the elution amounts of the respective ions per sample mass in the test sample 2-4 and the test sample 2-5.

TABLE 5

| | Ca | P |
|---|---|---|
| | (mg/g) | (mg/g) |
| Test sample 2-4 | 14 | 5.8 |
| (containing fluorine) | | |
| Test sample 2-5 | 16 | 6.2 |
| (not containing fluorine) | | |

As shown in Table 5, it was observed that the elution amounts of the respective ions were smaller in the test sample 2-4 than in the test sample 2-5. From this fact, it turned out that the test sample 2-4 containing fluorine has higher acid resistance than the test sample 2-5 that did not contain fluorine.

While the foregoing has described what are considered to be the best mode and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present teachings.

The entire contents of Japanese Patent Application No. 2019-200776 (filed on Nov. 5, 2019) and Japanese Patent Application No. 2019-200777 (filed on Nov. 5, 2019) are incorporated herein by reference.

1. A glass structure comprising:
a plurality of glass particles, each of the glass particles including SiO₂, CaO and P₂O₅; and
a bonding portion that bonds the glass particles to one another and contains hydroxyapatite,
wherein at least a part of the hydroxyapatite is crystalline in the bonding portion, and
wherein a porosity of the glass structure is 15% or less.

2. The glass structure according to claim 1, wherein the bonding portion further contains apatite including fluorine.

3. The glass structure according to claim 1, wherein, in an X-ray diffraction pattern measured by a Cu-Kα ray, the glass structure contains a crystal phase having diffraction peaks at:
2θ=25.0° or more and 26.0° or less;
2θ=31.0° or more and 33.0° or less; and
2θ=39.0° or more and 40.0° or less.

4. The glass structure according to claim 1, wherein a composition ratio of SiO₂ in the glass particles is 20% by mass or more.

5. The glass structure according to claim 1, wherein a composition ratio of CaO in the glass particles is 20% by mass or more.

6. The glass structure according to claim 1, wherein a composition ratio of P₂O₅ in the glass particles is 50% by mass or less.

7. The glass structure according to claim 1, wherein the glass particles have an ability to form apatite.

8. A method for producing the glass structure according to claim, 1, the method comprising:
preparing a mixture by mixing a plurality of glass particles and an aqueous solution with each other, each of the glass particles including SiO₂, CaO and P₂O₅, and the aqueous solution including calcium and phosphorus and having pH of 4.0 or more; and
heating and pressurizing the mixture.

9. The method for producing the glass structure according to claim 8, wherein the aqueous solution further includes fluorine.

10. The method for producing the glass structure according to claim 8, wherein a temperature of heating the mixture is 40° C. or more and 300° C. or less.

11. The method for producing the glass structure according to claim 8, wherein a pressure to be applied to the mixture is 1 MPa or more.

12. The method for producing the glass structure according to claim 8, wherein a time of heating and pressurizing the mixture is 30 minutes or more.
